(19) United States
(12) Patent Application Publication
(10) Pub. No.: US 2011/0219520 A1
(43) Pub. Date: Sep. 15, 2011

(54) "Ice-N-wear" & "heat-N-wear" biker shorts

(76) Inventor: Edward J. Roland, Vallejo, CA (US)

(21) Appl. No.: 12/802,127

(22) Filed: Jun. 1, 2010

Related U.S. Application Data
(60) Provisional application No. 61/339,966, filed on Mar. 12, 2010.

(51) Int. Cl. A41D 1/08; A41D 27/20; A61F 7/00

(52) U.S. Cl. 2/228; 2/250; 607/112; 607/108

(57) ABSTRACT

The new features that my invention consist of on biker shorts is a (7″/13″) inside support hip pocket that will allow a (4″/10″) ice or heat pack be inserted on the left or right hip inside the biker shorts, that will be fasten by a 4½″/⅝″ Velcro strap. With this added dimension to biker shorts will give an individual an option to apply an ice or heat pack at their convenience or in the comfort of their home, work, gym etc.

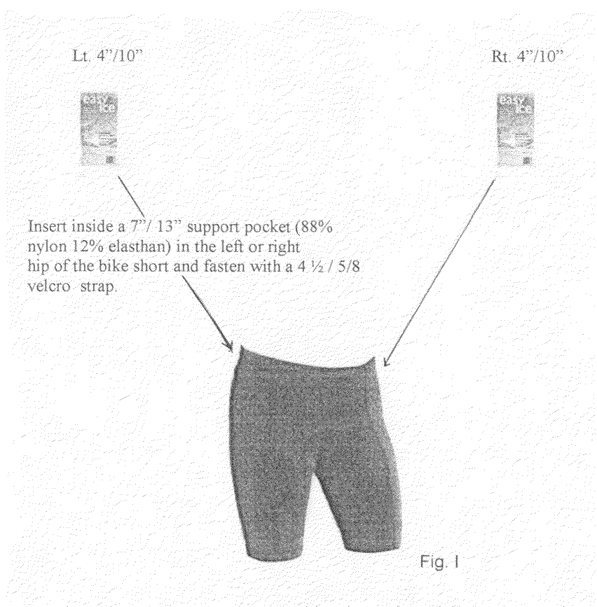

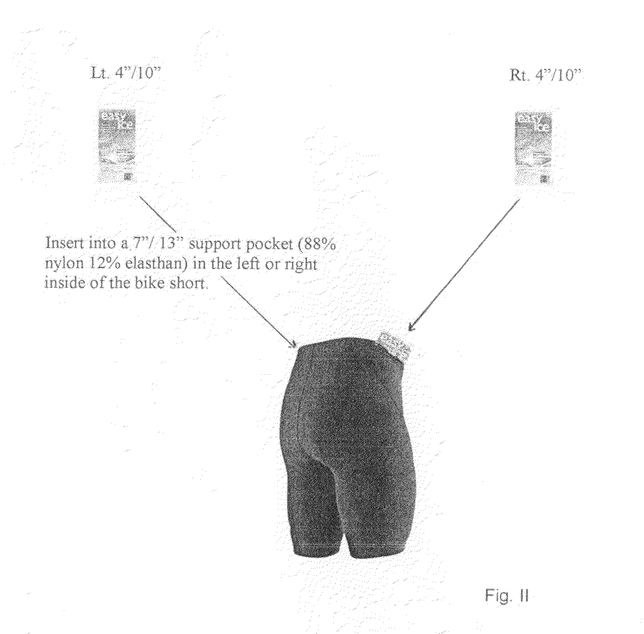

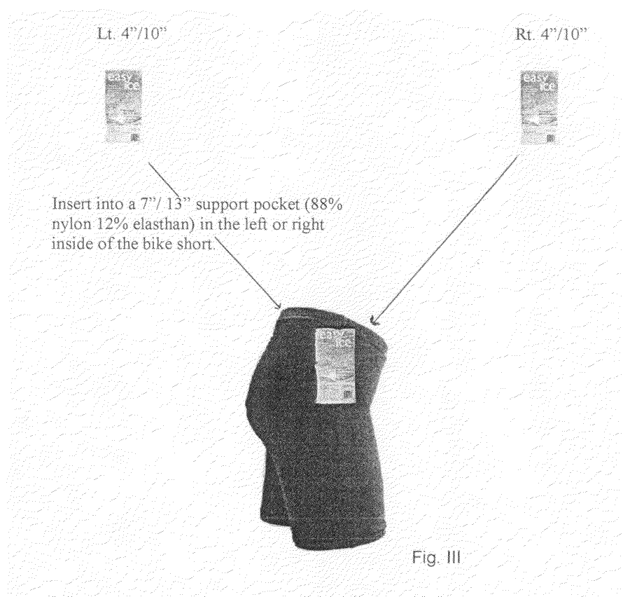

What I claim on my application is my provisional application Ser. No. 61/339,966 filed on Mar. 12, 2010.

### CROSS-REFERANCE TO RELATED APPLICATION

N/A

### STATEMENT REGARDING FEDERALLY SPONSORED RESEARCH OR DEVELOPMENT

N/A

### REFERANCE TO SEQUENCE LISTING

N/A

### SEQUENCE LISTING

N/A

### DETAILED DESCRIPTION OF THE INVENTION

“ice-N-wear” & “heat-N-wear” biker shorts was design to enhance an individual ability by maximizing his or hers work out to a certain level by inserting a (4″/10″) reusable, instant ice or heat pack on the left or right support hip pocket (7″/13″) inside the biker shorts. The inside support hip pocket is fasten by a 4½″/⅝″ Velcro strap which holds the (4″/10″) ice or heat pack in place for convenience. By doing so, this would make recovering from sore hip muscles, inflammation of the hip and arthritis of the hip a lot faster. Also the added features will allow the active, the athletically incline and the seasonal individual to maintain a consistent work out regimen that can be used before or after each work out.

### BACKGROUND OF THE INVENTION

“ice-N-wear” & “heat-N-wear” biker shorts are made from 88% polyester and 12% nylon 2-way stretched material. Stitched with 100% black polyester covered polyester thread. The (7″/13″) inside support hip pocket of the biker shorts is 80% nylon and 20% elasthan that is doubled stitched with 100% black covered polyester thread and fasten with a 4½″/⅝″ Velcro strap single stitched.

### BRIEF DESCRIPTION OF THE VIEWS OF THE DRAWINGS

In FIG. I drawing, is the front view of the biker shorts with two reusable ice or heat pack being displayed before insertion into the inside support hi pocket.

In FIG. II drawing, is a ¾ right side view of the biker shorts that is showing the ice or heat pack being inserted ¼ into the right inside support hip pocket.

In FIG. III drawing, is a right side view

1. What I claim as my invention is a (7″/13″) left and right support hip pocket inside of the biker shorts that is fasten by a 4½″/⅝″ Velcro strap that will hold the (4″/10″) reusable ice or heat pack in place.
